(19) United States
(12) Patent Application Publication
(10) Pub. No.: US 2012/0178822 A1
(43) Pub. Date: Jul. 12, 2012

(54) Co-Processed Excipient Compositions

(75) Inventors: Vinay Umesh Rao, Hyderabad (IN); Jagdish Balasubramaniam, Chennai (IN); Rama Krishna Haldar, Randolph, NJ (US)

(73) Assignee: ISP INVESTMENTS INC., WILMINGTON, DE (US)

(21) Appl. No.: 13/266,341

(22) PCT Filed: Apr. 26, 2010
(86) PCT No.: PCT/US10/32406

§ 371 Date: Mar. 30, 2012

Related U.S. Application Data
(60) Provisional application No. 61/175,558, filed on May 5, 2009.

(30) Foreign Application Priority Data
Apr. 28, 2009 (IN) 1118/MUM/2009

(51) Int. Cl. A61K 47/32

(52) U.S. Cl. 514/770

(57) ABSTRACT

An oral solid dosage form having improved dissolution profile and a method of producing the same are provided. The present invention particularly provides a co-processed excipient composition and a method of producing the same. More particularly, it relates to a co-processed binary mixture of crosslinked polyvinylpyrrolidone and calcium silicate; wherein the weight ratio of crosslinked polyvinylpyrrolidone and calcium silicate is in the range of 1:1 to 20:1. The binary mixture when combined with a poorly soluble drug enhances its dissolution and extent of release.

### FIELD OF THE INVENTION

The present invention relates to a co-processed excipient composition and to a method of producing the same. More particularly, it relates to a co-processed binary mixture of crosslinked polyvinylpyrrolidone and calcium silicate; wherein the weight ratio of crosslinked polyvinylpyrrolidone and calcium silicate is in the range of 1:1 to 20:1. The binary mixture when combined with a poorly soluble drug enhances its rate and extent of dissolution.

### DESCRIPTION OF THE PRIOR ART

A majority of drugs available today are poorly soluble and the poor water solubility is a challenge in pharmaceutical research. Relative to highly soluble compounds, low drug solubility often manifests itself in a host of in vivo consequences, including decreased bioavailability, increased chance of food effect, more frequent incomplete release from the dosage form and higher inter patient variability. Poorly soluble compounds also present many in vitro formulation obstacles, such as severely limited choices of delivery technologies and increasingly complex dissolution testing with limited or poor correlation to in vivo absorption.

In spite of the increased focus and interest generated in the area of controlled release and targeted drug delivery systems in recent years, tablet dosage forms that are intended to be swallowed whole, and which disintegrate and release their medicaments rapidly in the gastrointestinal tract still remain the formulation of choice from both a manufacturing and patient acceptability point of view. Thus, in any type of tablet system, the important variable is the rate at which the active substance goes in to solution or dissolves. Dissolution of the active substance is essential for it to be absorbed through the biological membranes into the systemic circulation for eliciting its desired pharmacological activity. For many solid dosage forms, disintegration occurs prior to drug dissolution and superdisintegrants are now frequently used in tablet formulations to improve the rate and extent of tablet disintegration and thus increase the rate of drug dissolution.

Ongoing research looks for better options which illustrate improvement in the processing of poorly soluble drugs and formulation techniques, which are not only simple and economic but also utilize the concepts of improving dissolution and thus overall bioavailability. U.S. Pat. No. 4,744,987 (Mehra et al) is directed to the use of a blend of microcrystalline cellulose (MCC) and calcium carbonate in pharmaceuticals for improving dissolution profile.

PCT WO 98/03064 assigned to FMC Corporation discloses a composition useful as an excipient for active agents in solid dosage forms which comprises a superdisintegrant and a co-disintegrant comprising diatomaceous earth, calcium silicate or a porous hydrophilic zeolite in an amount sufficient to provide rapid disintegration of the solid form when placed in solution.

EP 1923054 discloses a fast disintegrating solid pharmaceutical composition suitable for oral administration comprising a Sphingosine-1 phosphate receptor agonist and/or modulator. The ratio of the silicate, e.g. calcium silicate to disintegrant may be from 2:1 to 10:1, for example 3:1 to 7:1, typically 6:1, 5:1 or 4:1. However both components are added individually.

Further, US application Nos. 20080241234; 20070275059; 2007208069; and 20050019398 disclose the use of calcium silicate and a superdisintegrant in various ratios. However, these were added individually, to enhance disintegration time.

Another co-processed compressible composite of mannitol with calcium silicate is disclosed in PCT WO 2007/113856. The water soluble excipient and calcium silicate composites were prepared by co-processing for use in orally disintegrating tablet compositions.

It is well known that both calcium silicate and crospovidone are widely used excipients in pharmaceutical dosage forms, more particularly for oral solid dosage forms. Many of the prior references have disclosed the combined use of the superdisintegrant and co-disintegrants. However, the main aim of the combined use was to achieve enhanced disintegration time for fast disintegrating dosage form. Moreover, most of the references cited hereinabove disclose the use of calcium silicate and crospovidone as excipients amongst many others. In these instances they are added individually to the final composition.

In practicing the present invention, rapid disintegration rate is not a prerequisite for the dissolution. Thus, a focus of the present invention is to provide enhanced dissolution rate and extent of the drug from solid dosage forms, by utilizing co-processed excipient compositions comprising a binary mixture of crosslinked polyvinylpyrrolidone and calcium silicate.

According to the present invention, absorbent calcium silicate can be admixed with crospovidone and processed to form a binary mixture. When the binary mixture comes in contact with an aqueous medium, the water uptake is accentuated and as a result produces a synergistic effect in improving the dissolution rate and extent of release. The excipient composition of the present invention can be successfully used in conjunction with virtually all poorly soluble drugs.

Thus the present invention relates to an excipient composition comprising a co-processed binary mixture of crosslinked polyvinylpyrrolidone and calcium silicate useful for the formulation of a wide variety of poorly soluble drugs.

### SUMMARY OF THE INVENTION

The present invention provides a co-processed excipient composition comprising a co-processed binary mixture of crosslinked polyvinylpyrrolidone and calcium silicate. The weight ratio of crosslinked polyvinylpyrrolidone and calcium silicate preferably is in the range of 1:1 to 20:1, more preferably from about 5:1 to about 12:1

The co-processed binary mixture can be obtained by milling, spray drying or freeze drying.

The present invention also provides a composition wherein the binary mixture is further combined with the poorly soluble drug having solubility range from 2 microgram to 2300 microgram/ml, and the poorly soluble drug is selected from the group consisting of sparingly soluble, slightly soluble, very slightly soluble and practically insoluble drug substances.

The composition can be further formulated into an oral solid dosage form via dry granulation, wet granulation, direct compression, or the like. The co-processed binary mixture amounts to from about 0.1 to 10% of the total composition; more preferably from about 0.25 to 5 wt % of the total composition. Compositions of the present invention may be in the form of, for example, tablets, capsules, caplets, lozenges, pills, mini-tablets, pellets, beads and granules.

The oral solid dosage form of the present invention shows improvement both in the rate and extent of dissolution.

### BRIEF DESCRIPTION OF THE DRAWINGS

FIG. 1 is a Comparative Dissolution Profile of Ezetimibe with a co-processed binary mixture of crosslinked polyvinylpyrrolidone and calcium silicate (using ball mill/spray drying) and Control (calcium silicate and Crospovidone added separately).

FIG. 2 is a Comparative Dissolution Profile of Fexofenadine Hydrochloride with a co-processed binary mixture of crosslinked polyvinylpyrrolidone and calcium silicate (using ball mill) and Control (calcium silicate and Crospovidone added separately).

### DETAILED DESCRIPTION OF THE INVENTION

The co-processed excipient composition of the present invention provides greatly enhanced characteristics to poorly soluble drugs such as substantially increased rate and extent of dissolution.

The present invention relates to a co-processed excipient composition of disintegrants, a superdisintegrant, preferably polyvinylpyrrolidone, and an adsorbent, preferably calcium silicate.

The inventors have found that instead of using excipients separately or individually to produce certain effect, it is more advantageous to combine and formulate the excipients in such a way that they produce a synergistic effect. The present invention relates to the synergistic effect produced by the excipient composition which comprises a co-processed binary mixture of crosslinked polyvinylpyrrolidone and calcium silicate; wherein the weight ratio of the crosslinked polyvinylpyrrolidone and calcium silicate is in the range of 1:1 to 20:1. More preferably the ratio varies from about 5:1 to about 12:1.

The performances of dosage forms which comprise the binary mixture were compared with dosage forms where the excipients were added individually without using any formulation aid. The rate and extent of dissolution was greatly improved with the use of the co-processed binary mixture in various possible ratios. The observed synergistic effect is believed to be attributed to the intimate contact of calcium silicate and crospovidone due to co-processing via various means such as spray drying, milling, freeze drying and the like. The absorbent nature of the calcium silicate aids in improving the tendency of crospovidone to take-up water by wicking mechanism.

The co-processed excipient when combined with poorly soluble drugs and introduced into aqueous medium shows enhanced dissolution of the poorly-soluble drugs.

The term “co-processed excipient composition” as used herein, refers to a binary mixture of the two components, i.e., crosslinked polyvinylpyrrolidone and calcium silicate, that have been co-processed using various means such as co-milling, mechanical milling, spray drying, freeze-drying, etc. Milling is a preferred embodiment. The mechanically activated mill can be selected from the group consisting of a roller mill, a ball mill, a bead mill, a millstone mill, a jet mill, and a hammer mill.

In general, the term “poorly soluble drugs” refers to those drugs having solubility range from 2 microgram to 2300 microgram/ml of aqueous solubility. Particularly, poorly soluble drugs include drugs selected from the group consisting of sparingly soluble, slightly soluble, very slightly soluble and practically insoluble drugs. More particularly, the present invention is more particularly beneficial for the drugs selected from the group consisting of Indinavir, Rabeprazole, Stavudine, Venlafaxin, Cetirizine, Chlorpheniramine, Chlorpromazine, Divalproex, Losartan, Metoprolol, Metformin, Pantoprozole, Propranolol, Naproxen Na, Ranitidine, Amilodipine, Atenolol, APAP, Metolazone, Methycarbamol, Enalapril, Acyclovir, Azithromycine, Ciprofloxacin, Diclofenac, Esomeprazole, Fexofenadine, Flucanozole, Tizanidine, Terbinafine, Trimethoprim, Valsartan, Atorvastatin, Ibuprofen, Raloxifene, Tagesrod, Carbamazepine, Clopidogrel, Efaverinz, Ezetimibe, Itraconazole, Ketoconazole, Loratidine, Lovastatin, Simvastatin, Metaxalone.

The co-processed binary mixture of the present invention ranges from about 0.25 to 5 wt % of the total composition. In a preferred embodiment, the amount ranges from about 3% to about 5 wt %.

The term “crosslinked polyvinylpyrrolidone” as used herein, including the claims, refers to N-alkenyl-2-pyrrolidones; preferably it refers to 1-vinyl-2-pyrrolidone or more preferably to crospovidone. This class of polymer is well experimented with a wide range of drugs for improved performance and increased dissolution profile and are commercially available as Polyplasdone XL, Polyplasdone XL-10, and Polyplasdone INF-10, based on the particle size, sold by International Specialty Products (ISP).

Suitable further disintegrants, i.e., apart from the crosslinked polyvinylpyrrolidone mentioned above, are croscarmellose sodium, sodium starch glycolate, corn starch, potato starch, maize starch and modified starches, sodium stearyl fumarate, low substituted hydroxypropylcellulose and the like.

The term “calcium silicate” as used herein, including the claims, refers to silicate salt of calcium, also known as Calcium hydrosilicate, tobermorite, micro-cell, silene. Commercial products are generally prepared synthetically to help control their absorbing tendency and are made from lime and diatomaceous earth. Synthetic calcium silicate is a white or slightly cream colored, free-flowing powder, having the molecular formula CaSiO₃ and the CAS number 1344-95-2.

The excipient compositions are preferably prepared by physically mixing or milling or spray drying the crospovidone and the calcium silicate in the required ratio, preferably in a mechanically activated ball mill for around two hours. The speed is maintained at 200 rpm using approximately 25 stainless steel ball. This is followed by adding the excipient composition to the poorly soluble drug and formulating into an oral solid dosage form via dry granulation or wet granulation or direct compression.

In another aspect of the invention, other pharmaceutically acceptable excipients selected from the group consisting of diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, and chelating agents; used either alone or in combination.

The pharmaceutical compositions of the present invention are generally administered orally to patients, which include, but are not limited to, mammals, for example, humans, in the form of, for example, a tablet, a caplet, pills, capsules, granules or a suspension.

It will also be apparent to those skilled in the art that the pharmaceutical compositions of the present invention can be administered with other therapeutic and/or prophylactic agents and/or medicaments that are not medically incompatible therewith.

The following examples further illustrate the invention.

### Example 1

### Co-Processing of Crospovidone and Calcium Silicate

### Method 1: Milling

Required quantities of crosslinked polyvinylpyrrolidone and calcium silicate were weighed and co-milled in different ratios (Table 1) using a Retch ball mill instrument and milled for 2 hours using 25 stainless steel balls. After every 30 minutes the direction of rotation of the jar was programmed to change automatically.

### Method 2: Spray Drying

Required quantities of crosslinked polyvinylpyrrolidone and calcium silicate were weighed and mixed in different ratios (Table 1) in methanol to form a suspension, followed by drying the contents using a Buchi Spray dryer maintaining the flow rate of 10 ml/min; inlet temperature of 110° C.; outlet temperature 90° C. and aspiration rate of 80%-100%.

TABLE 1
Co-processed binary mixture of crosslinked
polyvinylpyrrolidone and calcium silicate

| Weight % crosslinked | |
|---|---|
| polyvinylpyrrolidone | Weight % calcium silicate |
| 50 | 50 |
| 80 | 20 |
| 75 | 25 |
| 90 | 10 |
| 95 | 5 |

### Example 2

### Ezetimibe Compositions with Crosslinked Polyvinylpyrrolidone and Calcium Silicate

Tablet formulations of Ezetimibe using co-processed binary mixture of crosslinked polyvinylpyrrolidone and calcium silicate were prepared by wet granulation using a Pro-C-Ept high shear granulator as per the formula given below:

TABLE 2
Tablet Formulation of Ezetimibe

| Ingredients | % by weight | mg/tablet |
|---|---|---|
| Ezetimibe | 10 | 10 |
| Lactose monohydrate | 38 | 38 |
| MCC | 87.5 | 87.5 |
| PVP K29/32 | 5 | 5 |
| SLS | 2 | 2 |
| Co-processed Crospovidone with Silicate* | — | 4.5 |
| Calcium silicate | 0.45 | — |
| Crospovidone | 4.05 | — |
| Magnesium Stearate | 1.5 | 1.5 |
| Aerosil | 1.5 | 1.5 |

*Prepared using Ball Milling/Spray drying

### Example 3

### Dissolution Studies

The dissolution studies of the prepared tablet as per Example 2 utilizing co-processed crospovidone with silicate and tablets comprising the similar ratio of calcium silicate and co-processed crospovidone added individually; were carried out using USP apparatus II (Venkel VK7010) with sinkers (5 spiral stainless steel capsule sinker of 18×6 mm capacity). A peristaltic pump was coupled to a Cary 50 UV/visible spectrophotometer to provide a continuous flow of drug solution through 0.2-cm cuvettes. Dissolution was performed in 1000 ml of water (phosphate buffer with 0.5% sodium lauryl sulphate; pH 7) at 37+0.5° C. Samples were programmed to be analyzed at 5, 10, 15, 30, 45 and 60 minutes at 285 nm.

TABLE 3
Comparative Dissolution Testing

| | | Tablet | Tablet |
|---|---|---|---|
| | Tablet | Co-processed | Co-processed |
| | Ca•Si:PVP* | Ca•Si:PVP* | Ca•Si:PVP* |
| | individually | via ball | via spray |
| Time(MIN) | added | milling | drying |
| 0 | 0 | 0 | 0 |
| 5 | 1 | 1 | 1 |
| 10 | 7 | 12 | 6 |
| 15 | 13 | 27 | 13 |
| 30 | 29 | 49 | 34 |
| 45 | 36 | 59 | 44 |
| 60 | 41 | 68 | 50 |

Ca.Si:PVP* = calcium silicate:crosslinked polyvinyl pyrrolidone

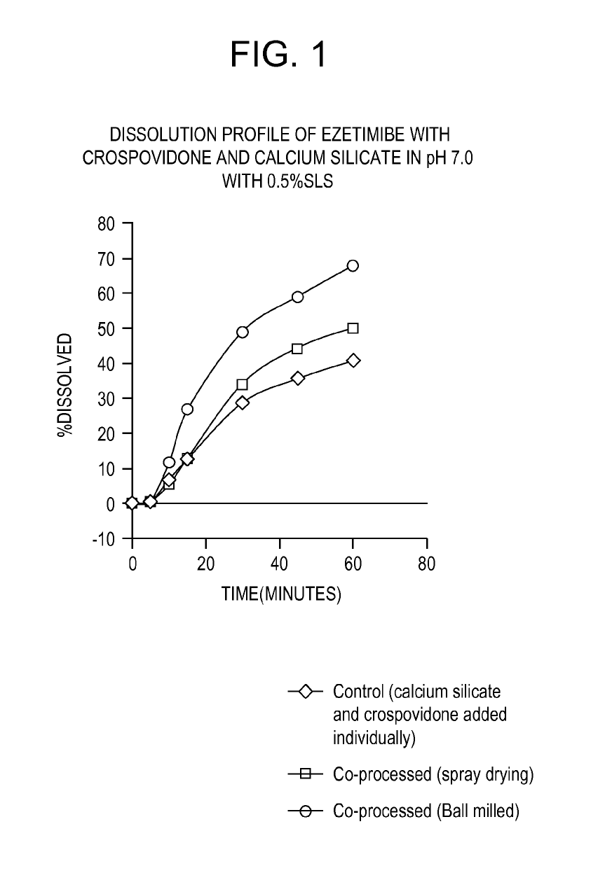

The results of dissolution testing, which are shown in FIG. 1, indicates that tablets prepared using a co-processed binary mixture of crospovidone and calcium silicate of the present invention gives better dissolution as compared to the excipient added individually. More particularly, co-processing done using ball milling has shown significantly greater dissolution as compared to the spray drying method.

### Example 4

### Fexofenadine Compositions with Crosslinked Polyvinylpyrrolidone and Calcium Silicate

Tablet formulations of Fexofenadine using a co-processed binary mixture of crosslinked polyvinylpyrrolidone and calcium silicate were prepared by wet granulation using a Pro-C-Ept high shear granulator as per the formula given below:

TABLE 4
Tablet Formulation of Fexofenadine Hydrochloride

| Ingredients | % by weight | mg/tablet |
|---|---|---|
| Fexofenadine HCl | 60 | 60 |
| MCC | 90 | 90 |
| Pregelatinized Starch (Dry) | 30 | 30 |
| Pregelatinized Starch | 10 | 10 |
| Co-processed Crospovidone with Silicate* | — | 6 |
| Calcium silicate | 0.6 | — |
| Polyplasdone XL | 5.4 | — |
| Magnesium Stearate | 2 | 2 |
| Aerosil | 2 | 2 |
| Total Tablet Weight | 200 | 200 |

The dissolution studies of the prepared tablets were carried out using USP apparatus II (Venkel VK7010) as described in Example 3.

TABLE 5
Dissolution Study

| | Tablet | Tablet |
|---|---|---|
| | Ca•Si:PVP* | Co-processed Ca•Si:PVP* |
| Time (min) | individually added | via ball milling |
| 0 | 0 | 0 |
| 5 | 46 | 59 |
| 10 | 62 | 75 |
| 15 | 70 | 82 |
| 30 | 82 | 92 |
| 45 | 86 | 97 |
| 60 | 90 | 100 |

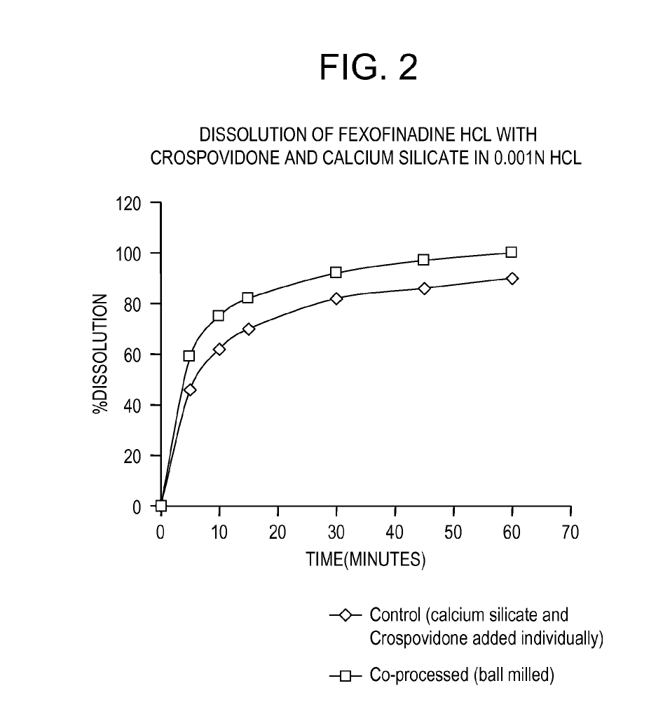

The results of Comparative Dissolution Profile of Fexofenadine Hydrochloride with co-processed binary mixture of crosslinked polyvinylpyrrolidone and calcium silicate (using Ball mill) and Control (calcium silicate and Crospovidone added separately) is illustrated in FIG. 2.

The invention has been described in detail with particular reference to preferred embodiments thereof, but it will be understood that variations and modifications can be effected within the spirit and scope of the invention.

What is claimed:

1. A co-processed excipient composition comprising a co-processed binary mixture of crosslinked polyvinylpyrrolidone and calcium silicate; wherein the weight ratio of crosslinked polyvinylpyrrolidone and calcium silicate is in the range of 1:1 to 20:1.

2. The excipient composition according to claim 1, wherein the range of crosslinked polyvinylpyrrolidone to calcium silicate is from about 5:1 to about 12:1.

3. The excipient composition according to claim 1, wherein said co-processed binary mixture is obtained by ball milling, spray drying or freeze drying.

4. The excipient composition according to claim 1, wherein said binary mixture is further combined with a poorly soluble drug having solubility range from 2 microgram to 2300 microgram/ml.

5. The composition according to claim 4, wherein the co-processed binary mixture amounts to 0.1 to 10 wt % of the total composition.

6. The composition according to claim 4, wherein the poorly soluble drug is selected from the group consisting of sparingly soluble, slightly soluble, very slightly soluble and practically insoluble.

7. The composition according to claim 4, wherein the composition further comprises excipients selected from the group consisting of diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, and chelating agents; used either alone or in combination.

8. The composition according to claim 4, wherein said composition is further formulated into an oral solid dosage form via dry granulation, wet granulation or direct compression.

9. The composition according to claim 4, wherein said oral solid dosage form shows improvement both in the rate and extent of dissolution.

10. A co-processed excipient composition and a process of its preparation as described and illustrated in the examples herein.
